(19) United States
(12) Patent Application Publication
(10) Pub. No.: US 2003/0103932 A1
(43) Pub. Date: Jun. 5, 2003

(54) Compositions, methods and devices for treatment of urethral disorders

(76) Inventors: Marvin J. Slepian, Tucson, AZ (US); Gill Heart, Tucson, AZ (US); Lucjan Hronowski, Bedford, MA (US)

Correspondence Address: Patrea L. Pabst, Esq., HOLLAND & KNIGHT LLP, 2000 One Atlantic Center, 1201 West Peachtree Street, Atlanta, GA, 30309-3400, US

(21) Appl. No.: 10/005,853

(22) Filed: Dec. 5, 2001

(51) Int. Cl. A61K 31/74; A61M 31/00

(52) U.S. Cl. 424/78.08; 604/500

(57) ABSTRACT

Compositions have been developed to reduce or relieve prostatic obstruction. The polymers are used as an endourethral polymer liner. A biodegradable polymer liner layer can be applied to the prostatic urethra by in situ casting, or insertion and shaping of preformed materials. This liner is preferably formed from structurally supportive, yet eventually biodegradable, polymers which further bolster and support the urethra and peri-urethral tissue during healing, eliminating the need for post-procedure catheter drainage. This step may be optional in specific clinical circumstances. Alternatively, the polymer coating may be applied to a structural material such as a stent, to decrease adhesion and/or provide release of drugs to enhance healing. The compositions can also be used for intra-prostatic void exclusion and space filling with adhesive and/or therapeutic polymers. Voids created as a result of intragland “shelling out” are filled with adhesive polymers which facilitate intraprostatic void cavity wall bonding and healing. Polymers are specifically selected to minimize inflammation, secondary bleeding and late fibrotic scarring and stricturing.

### BACKGROUND OF THE INVENTION

This invention relates to compositions, devices and methods for the removal and treatment of prostate tissue, using polymeric compositions to promote tissue involution, adhesion, thrombosis, decrease altered inflammation, and overproliferation of tissue, and optionally provide structural support and optionally deliver medicament for the same.

As men age, their prostate glands typically enlarge due to intra-gland growth of prostatic tissue (prostate adenoma) obstructing the flow of urine through the urethra. This condition is known as Benign Prostatic Hypertrophy (“BPH”), and results in a partial or total inability to urinate. There is a linear correlation of this disease with age. The incidence of BPH for men in their fifties is approximately 50%, rising to near 90% by age 85. About 25% of men in the United States will be under active treatment for BPH by age 80.

Traditional surgical therapy for BPH has involved open incision or transurethral resection of the prostate. Surgical treatment of BPH is generally reserved for patients with severe symptoms or for those who have developed urinary retention, renal damage caused by BPH, or those with significant potential complications if treatment were withheld. These painful procedures usually result in long-term recovery although the patient may be subjected to traumatic side-effects.

The most common surgical procedure, Transurethral Resection of the Prostate (“TURP”), involves the removal of the prostate's innermost core in order to enlarge the caliber of the prostatic urethra. The average TURP procedure costs approximately $12,000 and requires a hospital stay of approximately 3 to 4 days. During this period the patient is burdened with a Foley drainage catheter and bag. TURP side-effects include impotence (up to 30%), retrograde ejaculation, and short-term incontinence.

Suprapubic or Retropubic (Open) Prostatectomy (SPP/RPP) involves surgical removal of the enlarged prostate via an incision in the lower abdomen, usually requiring a 5 to 7 day hospital stay. Patients are allowed to return to work 2 to 3 weeks after the surgery. Open prostatectomy results in impotence (up to 30% of cases), retrograde ejaculation and incontinence.

Transurethral Incision of the Prostate (TUIP) is an endoscopic surgical procedure in which one to three cuts is made in the prostate to relax the constriction on the prostatic urethra. TUIP is limited to prostates below 30 grams and requires 2 days of hospitalization. TUIP patients may experience short-term incontinence, and rarely retrograde ejaculation.

Transurethral Vaporization of the Prostate (TUVP) is a procedure for ablating the prostatic tissues by vaporization using an electrosurgical roller. The cost and the hospital stay for this procedure is almost similar to that of the TURP. Although TUVP causes less bleeding than TURP, the impotence rates are not dissimilar.

In balloon dilation, a catheter with a high-pressure balloon at the end is inserted through the urethra and into the prostatic urethra. The balloon is then inflated to stretch the prostatic urethra and to enlarge its caliber. Clinical studies have demonstrated a high rate of obstructive recurrence. This therapy has largely been abandoned.

Laser assisted Prostatectomy includes two similar procedures, Visual Laser Ablation of the Prostate (“V-LAP”) and Contact Prostate (“C-LAP”). Typically, the procedure is performed in the hospital under either general or spinal anesthesia, and at least an overnight hospital stay is required. In V-LAP, the burnt prostatic tissue then necroses, or dies, and over four to twelve weeks is sloughed off during urination. In C-LAP, the prostatic and urethral tissue is burned on contact and vaporized. The major drawbacks to these procedures include their high cost equipment and high re-treatment rates.

TransUrethral Microwave Therapy (TUMT) is based on a catheter inserted into the urethra, on which a microwave antenna is situated at the level of the prostate. The urethra can be spared by cooling, but will otherwise be destroyed. Scarring of the prostatic tissue enlarges the urethral lumen. The drawback of this treatment is long catherization time (1-6 weeks) and high-retreatment rates.

TransUrethral Needle Ablation (TUNA) is performed by transurethrally inserting two radio-frequency antennas into the prostatic tissue for heat damage creation. The drawbacks involved are a long catherization period (up to 6 weeks) and very high re-treatment rates. Interstitial Laser Coagulation (ILC) is very similar to TUNA but the heat source is a laser.

High Intensity Focused Ultrasound (HIFU) brings a beam of ultrasound into a tight focus at a selected depth within the prostate, generating temperatures of 80-100° C. and causing coagulation necrosis. The energy is delivered transrectally, and a catheter is inserted into the urethra for enhancing the treatment. The drawbacks of this treatment is the major cost of the equipment and long catherization periods.

Water Induced Thermotherapy (WIT) is similar to non-urethra sparing microwave treatments. The heat damage is created by heating a balloon at the prostatic urethra and by heating the prostatic tissue. It has the same drawbacks as microwave treatments.

Holmium Laser Prostatectomy is comparable to open prostatectomy. During this treatment, as in open surgery, the entire hypertrophied gland is inoculated (but endoscopically) and dropped into the bladder. This gland should be morselated for removal. The drawbacks of this treatment are the cost of the equipment and the long learning curve.

In addition to the above, a few general limitations emerge regarding alternative therapies. By targeting tissue killing to regions surrounding the urethra, some relief of compressive urethral obstruction is achieved. However, with the exception of Holmium Laser Prostatectomy, none of these procedures directly removes material. All of these techniques rely on the body's response to injury and inflammation (the reticuloendothelial system (RES)) to slowly remove necrotic cells and “clean-up” the area. As such, all of these techniques take several months to ultimately lead to a maximal effect, which is also limited. In many of these techniques no actual net tissue removal or reduction occurs. Rather, the injury may lead to localized scarring and fibrosis which may ultimately lead to obstruction recurrence. The response to injury is individually variable and lesser degrees of relief are often achieved. Patients who are treated by thermotherapy typically recover quickly, but need to be catheterized for at least one week post-treatment to maintain urine flow. Even after catheter removal, irritating urinary symptoms frequently persist during the period of tissue sloughing and healing.

Drug therapy is sometimes an option. Some drugs are designed to shrink the prostate by inhibiting or slowing the growth of prostate cells. Other drugs are designed to relax the muscular tissue in the prostate capsule and bladder neck to relieve urethral obstruction. Current drug therapy (including Finasteride Therapy, Alpha Blocker Therapy and Phytotherapy) generally requires daily administration for the duration of the patient's life, and are known to cause dizziness and fainting, decreases in blood-pressure, impotence, retrograde ejaculation or a reduction in the volume of ejaculated sperm. Furthermore, the effectiveness of these drug therapies in long-term treatment of BPH has not been proved scientifically.

To date, the most effective surgical intervention for BPH is TURP. This procedure is invasive, requiring general anesthesia, several days of hospitalization and post-treatment placement of a drainage catheter. TURP is accompanied by significant bleeding with delayed passage of clots in the urine, significant pain and inconvenience. TURP frequently presents a high operative cost and risk for many patients. The potential disadvantages and limitations of TURP therefore include bleeding, urinary tract infections, urethral irritation, discomfort, occasional urinary incontinence, and sexual dysfunction. Despite these limitations, TURP is currently the gold standard of therapy for BPH.

It is therefore an object of the present invention to provide compositions, devices, and methods for improved treatment of BPH and all alternative invasive and minimally invasive therapies using alternative energy means, improving treatment outcome, reducing morbidity and complications and saving hospitalization and associated costs.

It is a further object of the present invention to provide polymeric materials, drugs and biologically active compositions which can be delivered or released within or adjacent to prostatic or urethral tissue to control bleeding and swelling and aid in healing.

### SUMMARY OF THE INVENTION

Compositions have been developed to reduce or relieve prostatic obstruction. The polymers are used as an endourethral polymer liner. As a final step, a biodegradable polymer liner layer is applied to the prostatic urethra by in situ casting, or insertion and shaping of pre-formed materials. This liner is preferably formed from structurally supportive, yet eventually biodegradable, polymers which further bolster and support the urethra and peri-urethral tissue during healing, eliminating the need for post-procedure catheter drainage. This step may be optional in specific clinical circumstances. Alternatively, the polymer coating may be applied to a structural material such as stent, to decrease adhesion and/or provide release of drugs to enhance healing. The compositions can also be used for intra-prostatic void exclusion and space filling with adhesive and/or therapeutic polymers. Voids created as a result of intragland “shelling out” are filled with adhesive polymers which facilitate intraprostatic void cavity wall bonding support and healing. Polymers are specifically selected to minimize inflammation, secondary bleeding and late fibrotic scarring and stricturing.

### BRIEF DESCRIPTION OF THE DRAWINGS

FIGS. 1a, 1b, and 1c are schematics of polymer forms for in situ formation of endoprostatic tubular non-cylindrical stents.

FIGS. 2a, 2b, 2c, 2d, 2e, and 2f are polymer sheets that can be used to form stents. FIG. 2a, solid; FIG. 2b, have pores or interstitial spacings therein; FIG. 2c, having spiral ribs on one or both sides of the sheet; FIG. 2d, having long axial ribs on one or both sides of the sheet; FIG. 2e, having short axial ribs on one or both sides; and FIG. 2f, mesh.

FIGS. 3a, 3b and 3c are schematics of different methods for making a seam in the polymeric sheets. FIG. 3a is where overlapping or abutting polymer is melted together; FIG. 3b is where another polymer fuses the polymer edges together; and FIG. 3c is where an adhesive is used to glue the edges together.

FIGS. 4a and 4b are prospective views of devices for use in deploying polymeric coatings at a site of injury. FIG. 4a shows an expandible element such as a balloon, having a polymer sheet wrapped around it, which is inserted into the lumen within a covering sheath. FIG. 4b is a prospective view of the expandible element of FIG. 4a, showing the center opening for a guide and insertion wire, heating or activating element for shaping the polymer sheet when the expandible element is expanded, and a sensor or detection device providing feedback during insertion and expansion.

FIGS. 5a-5g are prospective views of expandible elements for use in expanding and shaping a polymer sheet at the site of deployment.

### DETAILED DESCRIPTION OF THE INVENTION

I. Removal of Tissues and Application of Polymeric Material

There are several procedures that can be used to remove inflamed or enlarged prostate tissue, as discussed above, cancerous tissue, or tissue to provide relief from chronic urethritis or strictural disease. Following tissue removal, it is desirable to insert a stent to prevent reclosure of the urethra, to limit post surgical bleeding, decrease inflammation, to provide for controlled drug delivery of chemotherapeutic agents, antibiotics, and/or antiinflammatories, and/or to provide mechanical support.

The polymers are preferably selected to facilitate healing, with minimal inflammatory and late fibrotic responses, and can optionally be used for drug delivery of agents which further enhance the healing response. A biodegradable polymeric liner (typically formed of a different polymer system) and/or a non-polymeric stent with a suitable polymeric coating is placed within HMT 101 the lumen of the urethra to act as a wall support, maintaining urethral patency during healing, preventing patient discomfort and outflow obstruction.

Intraluminal and other spatial voids created as a result of endoprostatic procedures can also be filled with a material such as bioadhesive polymers which facilitate intraprostatic void cavity wall bonding and healing. Polymers are specifically selected to minimize inflammation, secondary bleeding and late fibrotic scarring. Material in a fluid state, or optionally in a solid state, can then be injected into the space, where the fluid and the material acts to fill the space. Alternatively, the material is exuded into the tissue cracks and crevices, then within the central lumen of the prostate. In a preferred embodiment, an adhesive polymer which may contain drugs for local prostate medication is injected into the cavity.

Polymer can also be used as an endomural support to prevent voided lobe collapse, thus widening the caliber of the prostatic urethra for non-obstructed voiding. The endomural support can be formed as an infusion into the cavity of polymer in a particulate form, in a liquid carrier, or where the polymer is preformed as a solid but in a chopped form as particles, flakes or fibers. The polymer (or polymerizable monomers or macromers) is applied liberally to the tissue surface, where it can conform and/or penetrate the surface. This is then either heated or polymerized to solidify the polymer, for example, by exposure to light, preferably while continually applying pressure which then expresses the fluid from the site of the tissue removal. The polymer may be configured by intra-urethral remolding or external, extraprostatic molding or both. A conventional stent can be used to support the tissue walls during this process. Pressure can be applied following application of polymeric material in the cavity by inflating a balloon in the urethra which compresses and closes the void around the inserted material, which acts as a glue-like or adherent material, thereby obliterating the void.

As discussed below, the polymeric material can include a therapeutic agent which then leads to further shrinkage or the bonding or modification of function of the tissue or its glandular hormonal production, as discussed in more detail below.

As a final step, a biodegradable polymer liner layer is applied to the prostatic urethra, preferably by in situ casting, although this can also be applied using a stent or catheter, either applied from the surface of the catheter or stent or applied from a reservoir in the catheter. This liner is formed from structurally supportive, yet eventually biodegradable, polymers, and supports the urethra and peri-urethral tissue during healing, elimination the need for post-procedure catheter drainage.

II. Selection and Application of Polymeric Materials and Drugs

1. Application and Configuration of Polymeric Materials

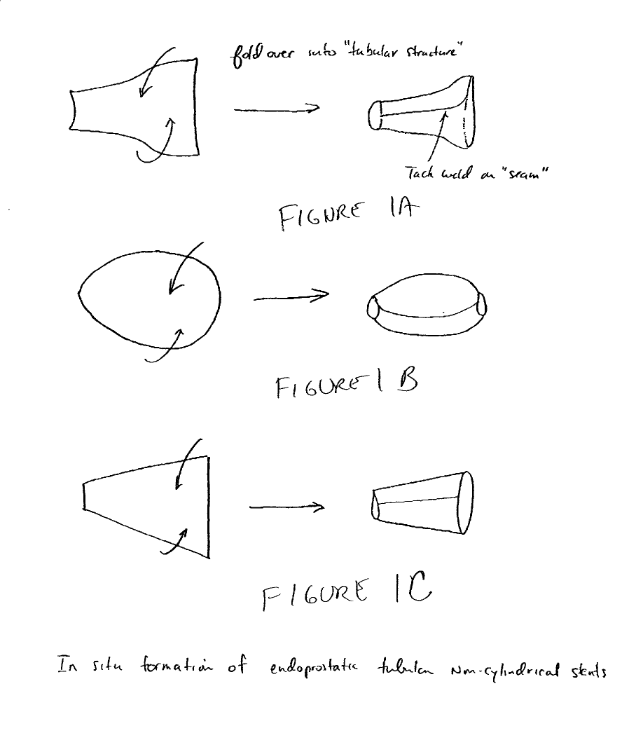

In the preferred embodiment, polymeric materials are applied to the surface of the tissue as a polymeric sheet. The form of the sheet is selected to go into the urethra where it then forms a non-tubular, complex shape, for example, a pear, spherical or combination shape. Representative shapes are shown in FIGS. 1a-1b. In FIG. 1a, the sheet is pear shaped, then folded after insertion, and tacked at the seam to form the desired shape. In FIG. 1b, the sheet is oval, and is folded and tacked in situ to form a convex shape. In FIG. 1c, the sheet is a trapezoid, and is folded in the urethra to form a conical structure.

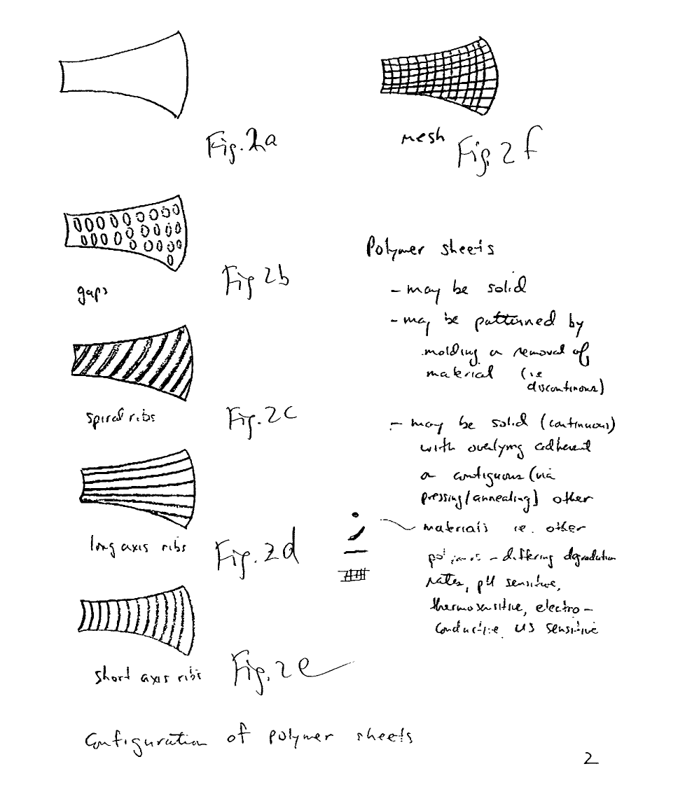

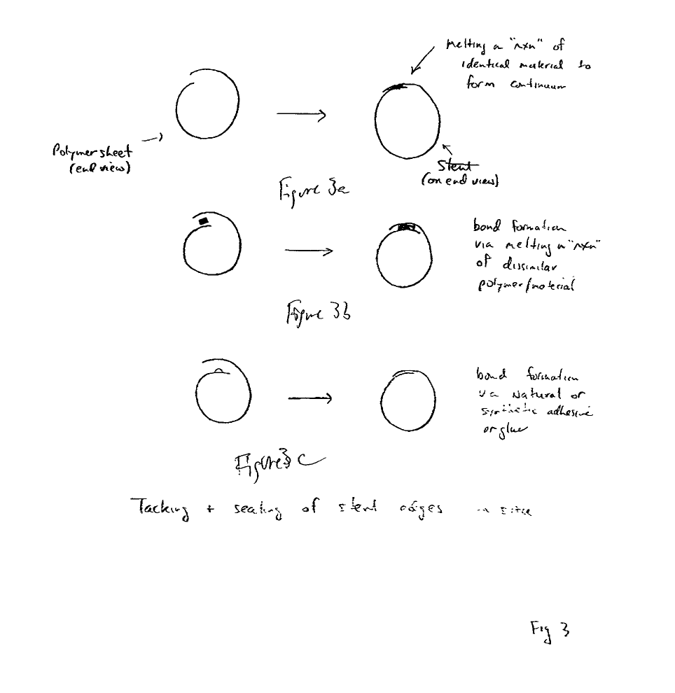

As shown in FIGS. 2a-2f, the polymer sheets can be configured to provide specific properties. For example, while in FIG. 2a, the sheet is a smooth surface, FIG. 2b has pores or interstitial spacings through which cells or fluids can migrate, and FIG. 2f is a mesh, also having openings through which cells or fluids can move. FIGS. 2c and 3 show polymeric forms having structural elements. These can be formed into the polymeric sheet by the extrusion, casting or molding process, to create spiral ribs, ribs running the longitudinal axis of the sheet, or ribs running along the horizontal axis, or they can be created by using different polymeric materials to form the sheet. For example, the sheets may consist of alternating regions of different polymeric materials, some of which may have long degradation times alternating with others with short degradation times. Some regions may be pliable and elastic, others rigid and providing mechanical strength.

Additional support pieces can also be inserted onto or under the polymer sheets. These may consist of support pieces, mesh, or braids.

The edges of the sheets can be secured as depicted in FIGS. 3a-3c. In FIG. 3a, the polymer is melted together. In FIG. 3b, another polymeric material or adhesive is melting or ultrasonically glued together. In FIG. 3c, a natural or synthetic adhesive or glue is used to adhere the edges together.

The liner can then be shaped to conform to the tissue surface. This can be accomplished by heating, pressure, ultrasound, or use of an adhesive on the tissue surface, either of the entire polymeric sheet or spots or regions thereof.

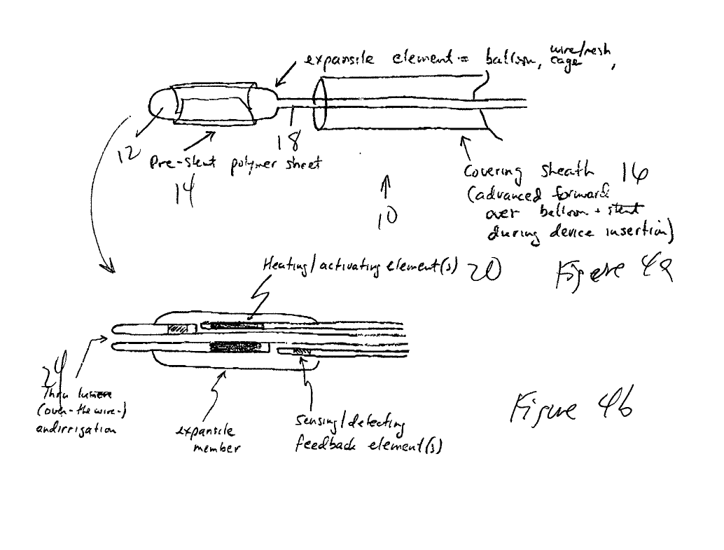

FIGS. 4a and 4b are prospective views of devices for use in deploying polymeric sheets as described above to form coatings at a site of injury. FIG. 4a shows a device 10 including an expandible element 12 such as a balloon, having a polymer sheet 14 wrapped around it, which is inserted into the lumen within a covering sheath 16 using a guide wire 18. FIG. 4b is a drawing of the expandible element 12 of FIG. 4a, showing the center opening for a guide and insertion wire 18 which is inserted into the lumen 24, heating or activating element 20 for shaping the polymer sheet 14 when the expandible element 12 is expanded, and a sensor or detection device 22 providing feedback during insertion and expansion. There may be multiple lumens 24 through which the wire may be inserted, for inflow/outflow of lubricants, washing fluids, medicines, etc.

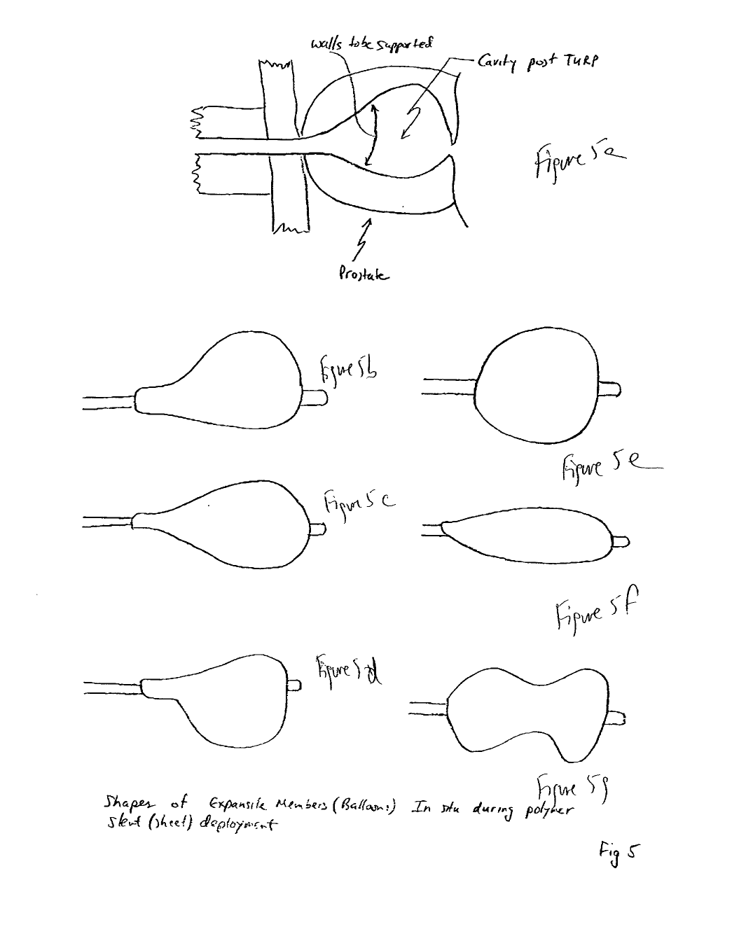

The heating or activating element can be used to shape or alter the physical properties of the polymer sheet. For example, the element can generate heat by means such as conductive heat, resistance heating, radiofrequency heating, microwave heating, electrochemical heating, light absorbance or generation, infrared, fiberoptics, or ultrasound. The expandible element will usually be formed of a material such as an elastomer such as latex, C-Flex™, butadiene, silicone rubber, and other natural or synthetic polymers. FIGS. 5a-5g show representative shapes of expandible elements.

The sensing means is typically used for feedback and temperature control.

These devices can further include means for deployment, such as means for opening, unfurling, tacking or securing, of the polymer sheets at the site of injury.

In an optional embodiment, polymeric materials are applied at the surface of or interior of cavities created by removal of prostatic tissue, and/or in the urethra to prevent obstruction due to overproliferation or inflammation of the adjacent tissue resulting from the surgical treatment. These materials can be used to adhere the sides of the tissue cavity together, to form a barrier at the surface of one or more of the tissue surfaces, for delivery of bioactive agents, for the retention of radioisotopes, radioopaque particulate, etc. The polymer may be deployed in the interior of the endomural tissue of the vessel or organ from the surface or tip of the catheter. Alternatively, the polymer can be applied by spraying, extruding or otherwise internally delivered via a long flexible tubular device consisting of as many lumens as a particular application may dictate. The coating typically will be applied to a tissue surface using some type of catheter. The material is preferably applied using a single catheter with single or multiple lumens. The catheter should be of relatively low cross-sectional area. A long thin tubular catheter manipulated using direct using direct visual, ultrasound or fluoroscopic guidance is preferred for providing access to the interior of organ areas.

Application of the coating material may be accomplished by extruding a solution, dispersion, or suspension of monomers, polymers, macromers, or combinations thereof through a catheter to coat or fill a tissue surface or cavity, then controlling formation of the coating by introducing crosslinking agents, gelling agents or crosslinking catalysts together with the fluent material and then altering the conditions such that crosslinking and/or gelling occurs. Thus, when a balloon catheter is used, a flow of heated or chilled fluid into the balloon can alter the local temperature to a level at which gelling or cross-linking is induced, thereby rendering the material non-fluent. Localized heating or cooling can be enhanced by providing a flow of heated or chilled liquid directly onto the treatment site. Thermal control can also be provided, however, using a fluid flow through or into the balloon, or using a partially perforated balloon such that temperature control fluid passes through the balloon into the lumen. Thermal control can also be provided using electrical resistance heating via a wire funning along the length of the catheter body in contact with resistive heating elements. This type of heating element can make use of DC or radio frequency (“RF”) current or external RF or microwave radiation. Other methods of achieving temperature control can also be used, including light-induced heating using an internal fiber (naked or lensed). Similar devices can be used for application of light, ultrasound, or irradiation.

An advantage of the polymeric materials is that they can be tailored to shape the polymer into uneven surface interstices, while maintaining a smooth surface with good flow characteristics. Preferably the method utilizes biodegradable or bioerodible synthetic or natural polymers, with specific degradation, life span and properties, which can be applied in custom designs, with varying thicknesses, lengths, and three-dimensional geometries (e.g. stellate, linear, cylindrical, arcuate, spiral 8, etc.).

The pharamceutical delivery function of the process may be readily combined with the “customizable” deployment geometry capabilities to accommodate the interior of a myriad of complex organ or vessel surfaces. For example, polymer can be applied in either single or multiple polymer layer configurations and different pharmacological agents can be administered by application in different polymer layers when multiple polymer layers are used.

Polymer can also be used to coat the devices including the cutting device, stents, prosthetics, and catheters. Typically these coatings would be provided to minimize tissue reaction (such as adherence of tissue to the device or initiation of an inflammatory reaction) and/or for drug release.

The process of fixing the shape of the polymeric material can be accomplished in several ways, depending on the character of the original polymeric material. For example, a partially polymerized material can be expanded using a balloon after which the conditions are adjusted such that polymerization can be completed, e.g., by increasing the local temperature or providing UV radiation through an optical fiber. A temperature increase might also be used to soften a fully polymerized sleeve to allow expansion and facile reconfiguration and local molding, after which it would “freeze” in the expanded position when the head source is removed. Of course, if the polymeric sleeve is a plastic material which will permanently defer upon stretching (e.g., polyethylene, polyethylene terephthalate, nylon or polyvinyl chloride), no special fixation procedure is required.

2. Selection of Polymeric Materials

A variety of different materials can be used, depending on the purpose, for example, structural, adhesive, barrier, coating and/or drug delivery. As used herein, “polymer or polymeric material” includes materials other than polymers, such as macromers or monomers which polymerize to form polymers, as well as non-polymeric materials having the same function (for example, a bulking agent formed from hydroxyapatite is technically not a polymer but may be equally effective for filling in the cavity within the prostate or for release of drugs. The material may be in a solid or liquid form which can be converted to a solid form.

The nature of the polymeric material used will be a function of whether it functions as a coating, bandage, adhesive, drug delivery device, or mechanical support role. Further, the choice of polymer must appropriately balance the degree of structural and geometric integrity needed against the appropriate rate of biodegradation over the time period targeted to prevent an undesirable reaction. In some cases, the material may be the same for different purposes where the ultimate in vivo geometry of the polymer dictates the final function of the polymer coating. The thinner applications allow the polymer film to function as a coating, sealant and/or partitioning barrier, bandage, and drug depot. Complex internal applications of thicker layers of polymer may actually provide increased structural support and, depending on the amount of polymer used in the layer, may actually serve in a mechanical role to maintain vessel or organ potency. For example, obstructive tissue lesions which are comprised mostly of fibromuscular components have a high degree of viscoelastic recoil. This tissue requires using the process to apply an endoluminal mural coating of greater thickness and extent so as to impart more structural stability thereby resisting vessel radial compressive forces. This provides structural stability and is generally applicable for the maintenance of the intraluminary geometry of all tubular biological organs or substructure.

The polymeric materials can be applied as polymers, monomers, macromers or combinations thereof, maintained as solutions, suspensions, or dispersions, referred to herein jointly as “solutions” unless otherwise stated. Polymeric materials can be thermosettable, thermoplastic, polymerizable in response to free radical formation such as by photopolymerization, chemically or ionically crosslinkable (i.e., through the use of agents such as glutaraldehyde or ions like calcium ions). Examples of means of solidifying or polymerizing the polymeric materials including application of exogenous means, for example, application of light, ultrasound, radiation, or chelation, alone or in the presence of added catalyst, or by endogenous means, for example, a change to physiological pH, diffusion of calcium ions (alginate) or borate ions (polyvinyl alcohol) into the polymeric material, or change in temperature to body temperature (37° C.).

Materials can be selected for one or more properties, including bioadhesion, structural support or other biomechanical properties, controlled permeability (ranging from impermeable for barriers to selectively permeable to freely permeable), and having controlled, sustained or burst release of incorporated drugs. For those applications where structure is required, a polymer is selected which has appropriate mechanical and physical properties. Optimally, if a foreign support device or sealant material is to be introduced into the tissue, the polymeric coating used on the device should exert its intended effect principally during the period of healing and peak inflammatory reaction.

Although either non-biodegradable or biodegradable materials can be used, biodegradable materials are preferred for application to the cells or tissues. As used herein, “biodegradable” is intended to describe materials that are broken down into smaller units by hydrolysis, oxidative cleavage or enzymatic action under in vivo conditions, over a period typically less than one year, more typically less than a few months or weeks. For application to tissues to induce hemostasis, or prevent inflammation, enlargement and/or overproliferation, it is preferred to use polymers degrading substantially within six months after implantation. For prevention of adhesions or controlled release, the time over which degradation occurs should be correlated with the time required for healing, i.e., generally in excess of six weeks but less than six months, but may be a few days, weeks, or months. Tissue narrowing, if it does occur, tends to stabilize beyond the six month window following the initial procedure without further accelerated narrowing.

Suitable materials are commercially available or readily synthesizable using methods known to those skilled in the art. These materials include: soluble and insoluble, biodegradable and nonbiodegradable, natural or synthetic polymers. These can be hydrogels or thermoplastics, homopolymers, copolymers or blends, natural or synthetic. As used herein, a hydrogel is defined as an aqueous phase with an interlaced polymeric component, preferably with 90% of its weight as water. The preferred polymers are synthetic polymers, with controlled synthesis and degradation characteristics.

a. Representative Polymeric Materials for Direct Application to Tissue.

Representative natural polymers include proteins, such as zein, modified zein, casein, gelatin, gluten, serum albumin, or collagen, and polysaccharides, such as cellulose, dextrans, hyaluronic acid, polymers of acrylic and methacrylic esters and alginic acid. Synthetically modified natural polymers include alkyl celluloses, hydroxyalkyl celluloses, cellulose ethers, cellulose esters, and nitrocelluloses, acrylic or methacrylic esters of above natural polymers to introduce unsaturation into the biopolymers.

Representative synthetic polymers include polyphosphazines, poly(vinyl alcohols), polyamides, polycarbonates, polyalkylenes, polyacrylamides including poly(meth)acrylamides and derivatives thereof, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, polystyrene, polyvinyl pyrrolidone, and polyvinylphenol. Representative bioerodible polymers include polyhydroxyacids such as polylactides, polyglycolides and copolymers thereof, poly(ethylene terephthalate), poly(butic acid), poly(valeric acid), polycaprolactone, poly(lactide-co-caprolactone), poly[lactide-co-glycolide], polyanhydrides, polyorthoesters, blends and copolymers thereof.

These polymers can be obtained from sources such as Sigma Chemical Co., St. Louis, Mo., Polysciences, Warrenton, Pa., Aldrich, Milwaukee, Wis., Fluka, Ronkonkoma, N.Y., and BioRad, Richmond, Calif. or else synthesized from monomers obtained from these suppliers using standard techniques.

These materials can be further categorized as follows.

b. Materials Which Polymerize or Alter Viscosity as a Function of Temperature.

Poly(oxyalkene) polymers and copolymers such as poly(ethylene oxide)-poly(propylene oxide) (PEO-PPO) copolymers, and copolymers and blends of these polymers with polymers such as poly(alpha-hydroxy acids), including but not limited to lactic, glycolic and hydroxybutyric acids, polycaprolactones, and polyvalerolactones, can be synthesized or commercially obtained. For example, polyoxyalkylene copolymers are described by U.S. Pat. Nos. 3,829,506; 3,535,307; 3,036,118; 2,979,578; 2,677,700; and 2,675,619, the teachings of which are incorporated herein. Polyoxyalkylene copolymers are sold by BASF and others under the trade name Pluronics.TM. Preferred materials include F-127, F-108, and for mixtures with other gel materials, F-67. These materials are applied as viscous solutions at room temperature or lower which solidify at the higher body temperature. Another example is a low Tm and low Tg grade of styrene-butadiene-styrene block copolymer from Polymer Concept Technologies, C-flex™. Polymer solutions that are liquid at an elevated temperature but solid at body temperature can also be utilized. For example, thermosetting biodegradable polymers for in vivo use are described in U.S. Pat. No. 4,938,763 to Dunn, et al.

c. Materials Which Polymerize in the Presence of Divalent Ions.

Several divalent ions including calcium, barium, magnesium, copper, and iron are normal constituents of the body tissues and blood. These ions can be used to ionically cross link polymers such as the naturally occurring polymers collagen, fibrin, elastin, agarose, agar, polysaccharides such as hyaluronic acid, hyalobiuronic acid, heparin, cellulose, alginate, curdlan, chitin, and chitosan, and derivatives thereof cellulose acetate, carboxymethyl cellulose, hydroxymethyl cellulose, cellulose sulfate sodium salt, and ethylcellulose.

d. Materials that can be Cross Linked Photochemically, with Ultrasound or with Radiation.

Materials that can be cross linked using light, ultrasound or radiation will generally be those materials which contain a double bond or triple bond, preferably with an electron withdrawing substituent attached to the double or triple bond. Examples of suitable materials include the monomers which are polymerized into poly(acrylic acids) (i.e., Carbopols™), poly(acrylates), polyacrylamides, polyvinyl alcohols, polyethylene glycols, and ethylene vinyl acetates. Photopolymerization requires the presence of a photosensitizer, photoinitiator or both, any substance that either increases the rate of photoinitiated polymerization or shifts the wavelength at which polymerization occurs. Photoinitiation has advantages since it limits the thickness which can be polymerized to a thin membrane. The radiolysis of olefinic monomers results in the formation of cations, anions, and free radicals, all of which initiate chain polymerization, grafting and crosslinking and can be used to polymerize the same monomers as with photopolymerization. Photopolymerization can also be triggered by applying appropriate wavelength to a cyclo-dimerizable systems such as Coumarin. Alpha-hydroxy acids backbone can be activated to carbonium ion. COOH or NH2 functionality can be inserted that can be subsequently reacted to amine containing ligands

e. Materials that can be Cross Linked by Addition of Covalent Crosslinking Agents such as Glutaraldehyde.

Any amino containing polymer can be covalently cross linked using a dialdehyde such as glutaraldehyde, or succindialdehyde, or succindialdehyde, or carbodimide (“CDI”). Examples of useful amino containing polymers include polypeptides and proteins such as albumin, and polyethyleneimine. Peptides having specialized function, as described below, can also be covalently bound to these materials, for example, using crosslinking agents, during polymerization.

f. Enhancement of Muco or Tissue Adhesive Properties of Polymeric Materials

Polymers with free carboxylic groups, such as the acrylic acid polymers noted above, can be used alone or added to other polymeric formulations to enhance tissue adhesiveness. Alternatively, materials that have tissue binding properties can be added to or bound to the polymeric material. Peptides with tissue adhesion properties are discussed below. Lectins that can be covalently attached to polymeric material to render it target specific to the mucin and mucosal cell layer could be used. Useful lectin ligands include lectins isolated from a variety of plants which are commercially available.

The attachment of any positively charged ligand, such as polyethyleneimine, polylysine or chitosan may improve bioadhesion due to the electrostatic attraction of the cationic groups to the net negative charge of the mucus. A surfactant-like molecule bearing positive charge and a hydrophobic core would be compatible with the bilayer membrane. This molecule will distribute its core and the positive charge to minimize energy of interaction and hence will be more tissue adhesive. The mucopolysaccharides and mucoproteins of the mucin layer, especially the sialic acid residues, are responsible for the negative charge coating. Any ligand with a high binding affinity for mucin could also be covalently linked to the polymeric material.

g. Protein Materials

Polymeric materials can also be used as tissue adhesives. In the simplest form, fibrin is used. This has the advantage that it can be formed easily in situ using the patient's own blood or serum, by addition of thrombin and calcium chloride. The materials described above can also be used. Other polymeric tissue adhesives that are commercially available include cyanoacrylate glues, Gelatin-resorcin-formaldehyde (“GRF”), and polyethyleneglycol-poly (lactic acid and/or glycolic acid)-acrylates, both of which are applied as liquids and then photopolymerized.

h. Manipulation of Physical Properties of Polymeric Materials

The polymeric material can be designed to achieve a controlled permeability, either for control of materials within the cavity or into the tissue or for release of incorporated materials. There are basically three situations that the polymeric material is designed to achieve with respect to materials present in the lumen: wherein there is essentially passage of only nutrients (small molecular weight compounds) and gases from the lumen through the polymeric material to the tissue lumen surface; wherein there is passage of nutrients, gases and macromolecules, including proteins and most peptides; and wherein there is passage of nutrients, gases, macromolecules and cells. The molecular weight ranges of these materials are known and can therefore be used to calculate the desired porosity. For example, a macromolecule can be defined as having a molecular weight of greater than 1000 daltons; cells generally range from 600-700 nm to 10 microns, with aggregates of 30-40 microns in size.

3. Bioactive Agents

a. Selection of Bioactive Agents

A wide variety of bioactive agents can be incorporated into the polymeric material. These can be physically incorporated or chemically incorporated into the polymeric material. Release of the physically incorporated material is achieved by diffusion and/or degradation of the polymeric material; release of the chemically incorporated material is achieved by degradation of the polymer or of a chemical link coupling the peptide to the polymer, for example, a peptide which is cleaved in vivo by an enzyme such as trypsin, thrombin or collagenase. In some cases, it may be desirable for the bioactive agent to remain associated with the polymeric material permanently or for an extended period, until after the polymeric material has degraded and removed from the site. A particularly useful group of bioactive agents to incorporate will be prothrombootic agents including collagen, fibrin, fibrinogen, tissue factor, any of the clotting factors, or surface activating agents such as silicates or diatomaceous earth.

In the broadest sense, the bioactive materials can include proteins (as defined herein, including peptides generally construed to consist of less than 100 amino acids unless otherwise specified), saccharides, polysaccharides and carbohydrates, lipids, nucleic acids, and synthetic organic and inorganic materials, or combinations thereof.

Specific materials include antibiotics, antivirals, anti-toxins hemostatics or anti-hemostatics, antiinflammatories, both steroidal and nonsteroidal, antineoplastics, anti-spasmodics including channel blockers, steroids including androgens, estrogens, progestins, or inhibitors of any of these steroid compounds, modulators of cell-extracellular matrix interactions including cell growth inhibitors and anti-adhesion molecules, enzymes and enzyme inhibitors, anticoagulants, growth factors, DNA, RNA and protein synthesis inhibitors, anti-cell migratory agents, vasodilating agents, and other drugs commonly used for the treatment of injury to tissue. Examples of these compounds include angiotensin converting enzyme inhibitors, anti-thrombotic agents, prostacyclin, heparin, salicylates, thrombolycytic agents, anti-proliferative agents, nitrates, calcium channel blocking drugs, streptokinase, urokinase, tissue plasminogen activator (“TPA”) and anisoylated plasminogen-streptokinase activator complex (“APSAC”), GPIIb/IIIA antagonists, colchicine and alkylating agents, growth modulating factors such as interleukins, transformation growth factor beta and congeners of platelet derived growth factor, fibroblast growth factor, epidermal growth factor, hepatocyte scatter factor, monoclonal antibodies directed against growth factors, modified extracellular matrix components or their receptors, lipid and cholesterol sequestrants, matrix metalloproteases (“MMPs”), collagenase, plasmin and other agents which may modulate tissue vessel tone, function, arteriosclerosis, and the healing response to vessel or organ injury post intervention.

Additional materials include hormones for hormone replacement therapy and chemotherapeutic agents such as BCNU, radioactive agents, and antibodies to tumor antigens.

Materials such as attachment peptides (such as the FN cell-binding tetrapeptide Arg-Gly-Asp-Ser (“RGDS”)), selectin receptors and carbohydrate molecules such as Sialyl Le.sup.s, can be used which serve to attract and bind specific cell types, such as white cells and platelets. Materials such as fibronectin, vimentin, and collagen, can be used to non-specifically bind cell types to enhance healing. Other proteins known to carry functional RGD sequences include the platelet adhesion proteins fibrogen, vitronectin and von Willebrand factor, osteopontin, and laminin. Specific RGD peptides are described in U.S. Pat. Nos. 4,517,686 to Ruoslahti, et al., 4,589,881 to Pierschbacher, et al., 5,169,930 to Ruoslahti, et al, 5,149,780 to Plow, et al, 4,578,079 to Ruoslahti, et al., 5,041,380 to Ruoslahti, et al., and Pierschbacher and Ruoslahti, J Biol Chem 262(36), 17294-17298 (1987), Mohri, et al., Amer J Hem 37:14-19 (1991), Aumailley, et al. FEBS 291(1), 50-54 (1991), Gurrath, et al., Eur J Biochem 210, 911-921 (1992) and Scarborough, et al., J Biol Chem 268(2), 1066-1073 (1993). Laminin is a large adhesive glycoprotein found in basement membranes which promotes cell adhesion, migration, differentiation, and growth (Kleinmen, et al., J Cell Biochem 27:317-325 (1985); Kleinman, et al., Biochem 25:312-318 (1986); and Beck, et al., FASEB J 4:148-160 (1990). A nonapeptide CDPYIGSR as well as the pentapeptide YIGSR, from the B1 chain promote cell attachment and migration (Graf, et al., Cell 48:989-996 (1987), and Biochem 26:6896-6900 (1987)).

Cells can also be incorporated in the material. Examples of useful cells include progenitor cells corresponding to the type of tissue at the treatment location or other cells providing therapeutic advantages. For example, liver cells might be incorporated into the polymeric material and implanted in a cavity created in the liver of a patient to facilitate regeneration and closure of that loumen. This might be an appropriate therapy in cases where diseases (e.g. cirrhosis, fibrosis, cystic disease or malignancy) results in non-functional tissue, scar formation or tissue replacement with cancerous cells. Similar methods may be applied to other organs as well. Cells to be incorporated include prostatic stromal cells and/or fibroblasts or other mesenchymal cells to facilitate closure of tissue voids. Alternatively, glandular epithelial cells, either mature, developing, embryonic/fetal or genetically engineered, may be deposited. These may serve to alter regional or systemic physiology through endocrine or paracrine hormone or other mediator release.

b. Physical Incorporation of Bioactive Agents

In most cases, it is possible to physically incorporate the bioactive agent by mixing with the material prior to application to the tissue surface or within the cavity and polymerization or solidification. The material can be mixed into the monomer solution to form a solution, suspension or dispersion. In another embodiment, the bioactive agent can be encapsulated within delivery devices such as microspheres, microcapsules, liposomes, cell ghosts or psuedovirions, which in themselves affect release rates and uptake by cells such as phagocytic cells.

c. Chemical Incorporation of Bioactive Agents

Bioactive agents can be chemically coupled to the polymeric material, before or at the time of polymerization. Bioactive materials can also be applied to the surface of stents or catheters used in the procedures described herein, alone or in combination with the polymeric materials. Catheter and other device or implant bodies are made of standard materials, including metals such as surgical steel and thermoplastic polymers. Occluding balloons may be made from compliant materials such as latex or silicone, or non-complilant materials such as polyethylene terephthalate (“PET”). The expansible member is preferably made from non-compliant materials such as PET, PVC, polyethylene or nylon. If used, the balloon catheter portion may optionally be coated with materials such as silicones, polytetrafluoroethylene (“PTFE”), hydrophilic materials like hydrated hydrogels and other lubricous materials to aid in separation of the polymer coating.

Several polymeric biocompatible materials are amenable to surface modification in which surface bound bioactive molecules/ligands exhibit cellular binding properties. These methods are described by Tay, et al., Biomaterials 10, 11-15 (1989), the teachings of which are incorporated herein by reference.

Covalent linkages can be formed by reacting the anhydride or acid halide form of an N-protected amino acid, poly (amino acid) (2 to 10 amino acids), peptide (greater than 10 to 100 amino acids), or protein with a hydroxyl, thiol, or amine group on a polymer. Peptides can be covalently bound to polymeric material, for example, when the polymeric material is a polymer of an alpha hydroxy acid such as poly (lactic acid), by protecting the amine functionality on the peptide, forming an acid halide or anhydride of the acid portion of the polymer, reacting the acid halide or anhydride with free hydroxy, thiol, or amine groups on the polymer, then deprotecting the amine groups on the peptide to yield polymer having peptide bound thereto via esterfication, thioesterfication, or amidation. The peptide can also be bound to the polymer via a free amine using reductive amination with a dialdehyde such as glutaraldehyde.

The ester groups on a polyester surface can be hydrolyzed to give active hydroxy and carboxyl groups. These groups can be used to couple bioactive molecules. Polyanhydrides can be partially hydrolyzed to provide carboxyl groups. The resulting carboxyl groups can be converted to acid halides, which can be reacted with amino acids, peptides, or other amine containing compounds with binding properties and form an amide linkage. Polyesters and polylactones can be partially hydrolyzed to free hydroxyl and carboxyl groups. Alternatively, if the hydroxyl groups are primary or secondary hydroxyl groups, they can be oxidized to aldehydes or ketones, and reacted with amines via reductive amination to form a covalent linkage. Polyamides can be partially hydrolyzed to provide free amine and carboxylic acid groups. The amine group can then be reacted with an amino acid or peptide in which the amine groups have been protected, and the carboxyl groups have been converted to acid halides. Alternatively the amine groups on the polyamide can be protected, and the carboxyl groups converted to acid halides. The resulting acid halides can then be reacted directly with the amine groups on amino acids or peptides. Polyalcohols with terminal hydroxy groups can be appended with amino acids or peptides. The acid halides described above can also be reacted with thiol groups to form thoesters.

d. Fillers and Viscosity Modifying Agents

Any of the foregoing materials can be mixed with other materials to improve their physiological compatibility. These materials include buffers, physiological salts, conventional thickeners or viscosity modifying agents, fillers such as silica and cellulosics, and other know additives of similar function, depending on the specific tissue to which the material is to be applied.

N-octyl or butyl cyanoacrylate (histoacryl) Gelatin-poly (L-Glutamic acid)—NHS or Gelatin-poly (L-Glutamic acid) reacted with water soluble carbodidimide (“WSC”) Photoactivatable Gelatin and PET-DA

4. Application of a Hydrogel Liner on the Urethral Lumen Following the TUVOR Procedure.

An incision in the prostatic tumor mass is made and a specific volume of the tumor is excised. The cavity is treated as described in examples 2 and 3. A polymeric coating can then be applied to the urethral lining to prevent reclosure and/or provide structural support. A variety of materials can be applied.

The present invention will be further understood by the following non-limiting examples.

Example 1

### Formation of a Polymeric Lining.

a. A hydrogel, in this case a C-flex™ powder is mixed with 2 g of 25% Pluronic™ (F-127) solution in PBS. 0.02 g of Triton™ surfactant is added to stabilize the suspension. The final suspension is applied laparoscopically to the urethral lumen area by a deploying device equipped with an infrared source. The infrared source is fired and held in place for 30 seconds. This results in a C-flex™ coated urothelial lumen (with intermittent irrigation of PBS).

b. In another embodiment, the same suspension is used and same procedure is implemented except that the deploying device has a saline firing system that irrigates the area of the urothelium during the deployment period of C-flex™.

Modifications and variations of the methods and compositions described above will be obvious to those skilled in the art and are intended to e encompassed by the following claims.

We claim:

1. A method for treating prostate disease, chronic urethritis or strictural disease comprising
applying a polymeric material to the walls of the lower urinary tract, cavity or the urethra lining.

2. The method of claim 1 comprising administering an adhesive polymeric material within the cavity.

3. The method of claim 1 comprising administering a polymeric material which provides mechanical or structural support to the prostate or urethra.

4. The method of claim 1 wherein the polymeric material has controlled permeability.

5. The method of claim 3 wherein the polymeric material forms a lining or support structure within the urethra.

6. The method of claim 4 wherein the polymeric material forms a barrier effective to decrease inflammation of the urethra.

7. The method of claim 1 wherein the polymeric material further comprises agents selected from the group consisting of prophylactic, diagnostic and therapeutic agents.

8. The method of claim 1 further comprising providing a stent or catheter having the polymeric material forming a coating thereon.

9. The method of claim 1 wherein the polymeric material is biodegradable.

10. The method of claim 1 wherein the polymeric material is applied as a liquid and solidified in situ.

11. The method of claim 10 wherein the polymeric material is polymerized in situ.

12. The method of claim 1 wherein the polymeric material is configured by conductive heat, resistance heating, radiofrequency heating, microwave heating, electrochemical heating, light absorbance or generation, infrared, fiberoptics, ultrasound or a combination thereof to conform the polymeric material to the tissue surface.

14. The method of claim 10 wherein the polymeric material is applied from a catheter.

15. The method of claim 14 wherein the polymeric form is polymerized by application of light from the catheter.

16. A polymeric form for application to the walls of the lower urinary tract, cavity or the urethra lining using a device having an expandible element to conform the polymeric form to the walls.

17. The form of claim 16 wherein the form is a sheet.

18. The form of claim 17 wherein the sheet forms a cone, convex three-dimensional shape or pear shape.

19. The form of claim 16 where the sheet is a solid, mesh, or porous form.

20. The form of claim 16 further comprising regions or structures providing mechanical support or having different mechanical or chemical properties.

21. The form of claim 16 further comprising a bioactive agent.

22. The form of claim 21 wherein the bioactive agent is selected from the group consisting of hemostatics, antibiotics, chemotherapeutics, antiinflammatories, and cells.

23. A catheter system comprising a guide wire, an expandible element to deploy a polymeric form to the walls of the lower urinary tract, cavity or the urethra lining, a polymeric form, and a covering sheath for the expandible element.
